(19) United States
(12) Patent Application Publication
(10) Pub. No.: US 2016/0235461 A1
(43) Pub. Date: Aug. 18, 2016

(54) Surgical Guide Apparatus and Method of Use

(71) Applicant: Michael H. Sumko, Perry, UT (US)

(72) Inventor: Michael H. Sumko, Perry, UT (US)

(73) Assignees: Michael H. Sumko, Pery, UT (US); Michael R. Schramm, Perry, UT (US)

(21) Appl. No.: 14/990,158

(22) Filed: Jan. 7, 2016

(51) Int. Cl. A61B 17/88

(52) U.S. Cl. CPC A61B 17/8897; A61B 2017/00438

(57) ABSTRACT

The invention disclosed herein is a surgical guide apparatus (SGA) and method of use. The guide apparatus preferably comprises a collar connected to a guide tube. The guide apparatus is expressly adapted to mount to the finger of a surgeon and to facilitate the proper insertion of a K-wire into a patient without tearing the surgeon's glove or cutting the surgeon.

### FIELD OF THE INVENTION

The present invention relates to surgical guide apparatuses, and more especially to surgical guide apparatuses that are wearable on a users finger and facilitate the insertion of a K-wire, fixation device, or marking device (unless indicated otherwise hereinafter all of which shall be considered to fall within the definition of K-wire).

### BACKGROUND OF THE INVENTION

A Kirschner wire or K-wire is well known in the orthopedic surgery industry and is widely used in surgical fixation applications (see Appx A). Further, many guide devices are known and are used in the orthopedic surgery industry to facilitate the correct insertion or placement (location and orientation) of such K-wires. Examples of such devices are disclosed in the following US patents, US patent applications, and US statutory invention registration, all of which are expressly incorporated herein by reference: U.S. Pat. Nos. 4,901,711, 5,891,161, 8,361,102, 8,540,676, 8,545,531, 8,715,311, 8,728,111, 8,979,889, 9,089,378, 20020099309, 20080015591, 20080021312, 20090216242, 20090299416, 20100211078, 20100331893, 20110118792, 20110213432, 20120041447, 20130317559, 20140324057, 20140336664, 20150223849, and H1706.

### SUMMARY OF THE INVENTION

Current practice for inserting a K-wire in a blind application (i.e. an application wherein the K-wire is subdermally inserted into a bone but the surgeon cannot physically see the insertion site but rather must “feel” for the proper insertion site) is as follows: The surgeon, wearing one or more pair of rubber gloves, makes an incision in the patient in the general area of the intended insertion site of the patient (e.g. a shoulder, hip, or knee site). The surgeon then inserts his finger into the incision and through the flesh of the patient until the surgeon feels the bone with his finger. The surgeon then moves his finger along the bone until the surgeon determines that the proper insertion sites has been located. The surgeon then slides a K-wire along the finger that is positioned at the proper insertion site and presses, drives, or otherwise inserts the K-wire into the bone at the proper location and preferably in the proper orientation. However, K-wires are typically very sharp so as to facilitate their insertion into bone. The process of sliding the K-wire along a gloved finger sometimes results in a torn glove. If the tear exposes the surgeon's finger to the surrounding tissue of the patient (e.g. the finger is “double gloved” but the K-wire cuts through both layers of glove) the surgeon may unintentionally expose the patient to infection. Worse yet, if the K-wire cuts the surgeon's finger, the surgeon's wound and blood may be exposed to the patient's open tissue and may cause infection to both the patient and the surgeon.

The invention disclosed herein is a surgical guide apparatus (SGA) and method of use. The guide apparatus preferably comprises a collar connected to a guide tube. The guide apparatus is expressly adapted to mount to the finger of a surgeon and to facilitate the proper insertion of a K-wire into a patient without tearing the surgeon's glove or cutting the surgeon.

### DESCRIPTION OF DRAWINGS

In order that the advantages of the invention will be readily understood, a more particular description of the invention briefly described above will be rendered by reference to specific embodiments that are illustrated in the appended drawings. Understanding that these drawings depict only typical embodiments of the invention and are not therefore to be considered to be limiting of its scope, the invention will be described and explained with additional specificity and detail through the use of the accompanying drawings, in which:

FIG. 1 is a trimetric view of a first embodiment of the SGA;

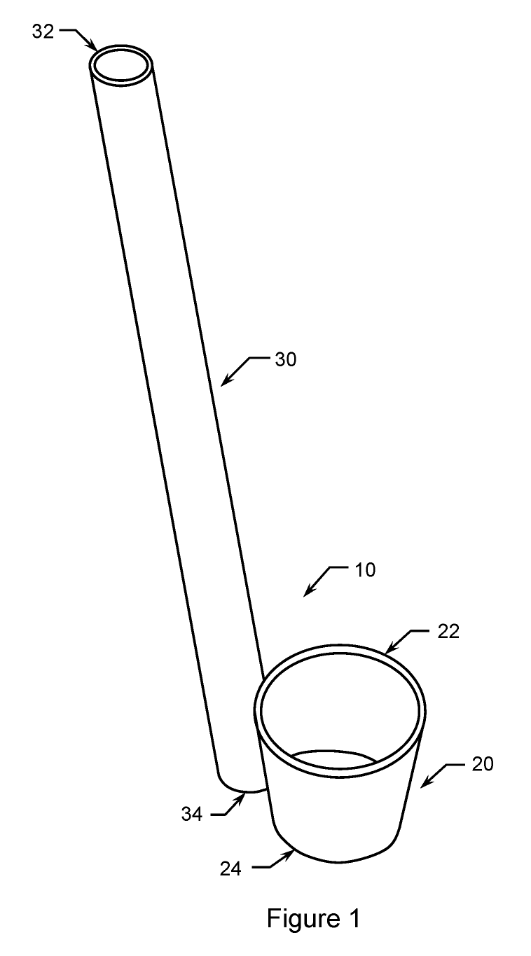

FIG. 2 is a trimetric view of a second embodiment of the SGA;

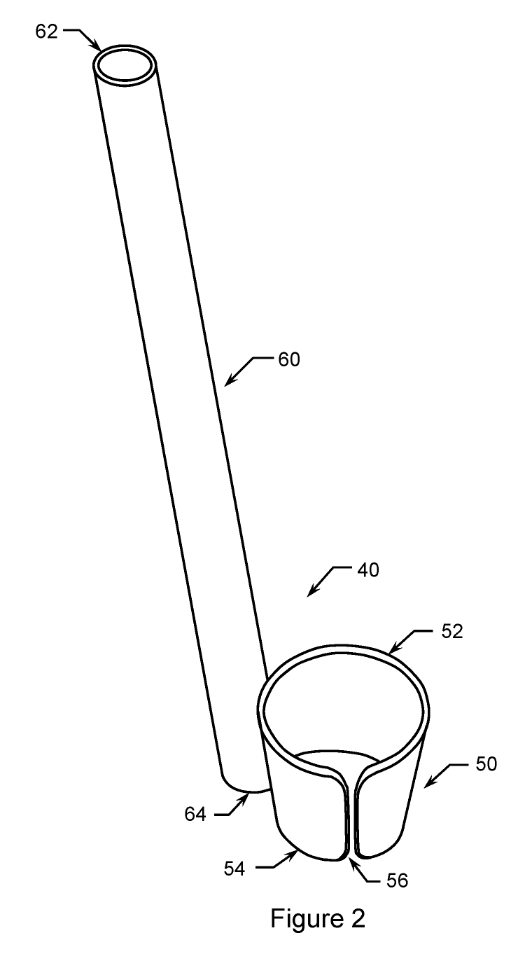

FIG. 3 is a trimetric view of the first embodiment of the SGA shown positioned or “worn” on a finger of a user (without showing a glove on the finger for the sake of clarity), and;

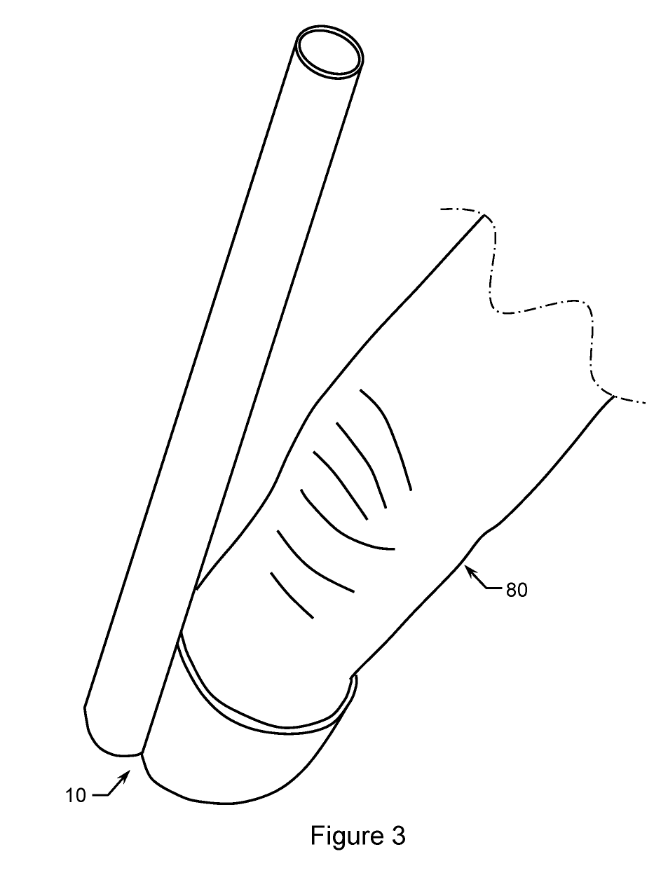

FIG. 4 is a side orthographic/sectional view of the first embodiment of the SGA shown positioned or “worn” on a finger of a user (without showing a glove on the finger for the sake of clarity) and with the user's finger positioned in an incision of a blind surgical position and with a K-wire prepared for insertion via the SGA.

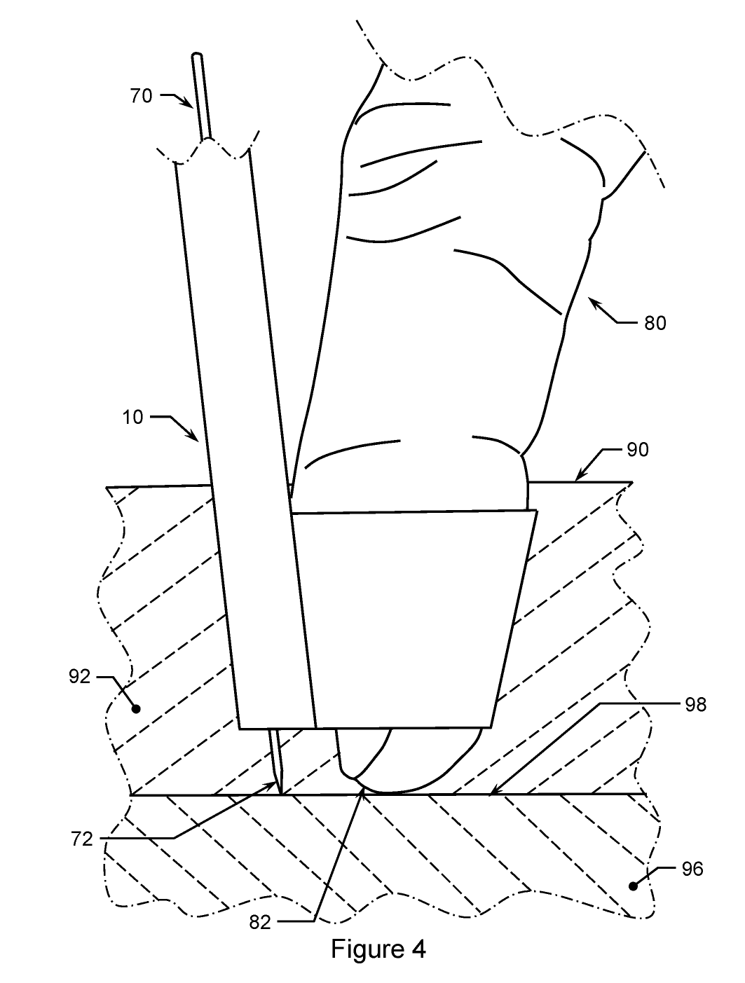

### DETAILED DESCRIPTION OF THE INVENTION

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

Furthermore, the described features, structures, or characteristics of the invention may be combined in any suitable manner in one or more embodiments. In the following description, numerous specific details are included to provide a thorough understanding of embodiments of the invention. One skilled in the relevant art will recognize, however, that the invention can be practiced without one or more of the specific details, or with other methods, components, materials, and so forth. In other instances, well-known structures, materials, or operations are not shown or described in detail to avoid obscuring aspects of the invention.

In order to facilitate the understanding of the present invention in reviewing the drawings accompanying the specification, a feature table is provided below. It is noted that like features are like numbered throughout all of the figures.

FEATURE TABLE

| # | Feature | # | Feature |
|---|---|---|---|
| 10 | Surgical guide apparatus | 20 | Collar |
| 22 | Collar upper end | 24 | Collar lower end |
| 30 | Guide tube | 32 | Tube upper opening |
| 34 | Tube lower opening | | |
| 40 | Surgical guide apparatus | 50 | Collar |
| 52 | Collar upper end | 54 | Collar lower end |
| 56 | Collar gap | 60 | Guide tube |
| 62 | Tube upper opening | 64 | Tube lower opening |
| 70 | K-wire | 72 | Sharpened tip |
| 80 | Finger | 82 | Fingertip |
| 90 | Skin | 92 | Subdermal tissue |
| 96 | Bone | 98 | Bone outer surface |

Referring now to the drawings, a first embodiment of the invention is a surgical guide apparatus (SGA) 10 for use in properly positioning and inserting a K-wire without causing damage to a user's gloves or hands comprising a collar 20 and a guide tube 30 connected to collar 20. Collar 20 preferably defines a generally hollow tapered cylindrical endless collar having an upper end 22 and a lower end 24. Guide tube 30 preferably defines a generally hollow cylindrical tube having an upper opening 32 and a lower opening 34. Collar 20 and guide tube 30 may be constructed of metal such as stainless steel and bonded, welded, fastened, or otherwise connected together. Alternative, collar 20 may be constructed of plastic and may be injection molded and guide tube 30 may be may be constructed of plastic and may be extrusion molded, with collar 20 and guide tube 30 bonded, welded, fastened, or otherwise connected together. Further alternatively collar 20 and guide tube 30 may both be constructed of plastic and may be injection molded as a single integral part. SGA 10 may be constructed as a reusable apparatus or as a single-use disposable apparatus. In practice, SGA 10 is preferably provided sterilized in a sealed container.

Referring now to the drawings, a second embodiment of the invention is a surgical guide apparatus (SGA) 40 for use in properly positioning and inserting a K-wire without causing damage to a user's gloves or hands comprising a collar 50 and a guide tube 60 connected to collar 50. Collar 50 preferably defines a generally hollow tapered cylindrical open-ended collar to provide flexure so as to provide for adjustable fit having an upper end 52, a lower end 54, and gap 56. Guide tube 60 preferably defines a generally hollow cylindrical tube having an upper opening 62 and a lower opening 64. Collar 50 and guide tube 60 may be constructed of metal such as stainless steel and bonded, welded, fastened, or otherwise connected together. Alternative, collar 50 may be constructed of plastic and may be injection molded and guide tube 60 may be may be constructed of plastic and may be extrusion molded, with collar 50 and guide tube 60 bonded, welded, fastened, or otherwise connected together. Further alternatively collar 50 and guide tube 60 may both be constructed of plastic and may be injection molded as a single integral part. SGA 40 may be constructed as a reusable apparatus or as a single-use disposable apparatus. In practice, SGA 40 is preferably provided sterilized in a sealed container.

In practice, a surgeon places collar 20 of SGA 10 (or alternatively collar 50 of SGA 40 ) on a gloved finger 80 to achieve a snug retainable fit of SGA 10 on finger 80, preferably with a small amount of fingertip 82 extending somewhat beyond collar lower end 24. With SGA 10 mounted on finger 80, the surgeon inserts finger 80 into an incision in a blind surgical application in a patient in need of orthopedic surgical intervention. The surgeon moves finger 80 while feeling for the proper insertion location until the proper insertion location is found. The surgeon then moves finger 80 by a small offset amount in the opposite of the direction of the center of tube lower opening 34 positioned from the center of collar lower end 24. For instance, if the distance from the center of tube lower opening 34 to the center of collar lower opening 24 is 0.25 inches, and if guide tube 30 is positioned to the right of collar 20, then the surgeon moves fingertip 82 by 0.25 inches to the left along bone outer surface 98, such that fingertip 82 is no longer directly adjacent the proper insertion location but approximately 0.25 inches away from the proper insertion location and the center of tube lower opening 34 is adjacent the proper insertion location. While maintaining tube lower opening 34 in proximate contact to the proper insertion location, the surgeon places K-wire 70 into guide tube 30 such that sharpened end 72 comes into contact with bone surface 98 at the proper insertion location. The surgeon then inserts K-wire 70 some distance into bone 96 with minimal risk of injury or damage to finer 80 or any gloves wore thereon. Such insertion of K-wire 70 into bone 96 may then function as an anchor for traction, as a fixation device itself, or as a marker around which the surgeon may use a cannulated (hollow) drill and drill a larger hole into bone 96 in preparation for insertion of a larger fixation device.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A surgical guide apparatus comprising a collar expressly adapted to mount to a user's finger, and a pin guide device connected to said collar.

2. The apparatus of claim 1, wherein said pin guide device further defines a hollow tubular pin guide device.

3. The apparatus of claim 1, wherein said collar is expressly adapted to press fittingly mount to a user's finger.

4. The apparatus of claim 1, wherein said apparatus includes a pin movably positioned within said pin guide device.

5. The apparatus of claim 4, wherein said pin defines at least one of a K-wire, a fixation device, and a marking device.

6. The apparatus of claim 1, wherein said apparatus is mounted to a user's finger.

7. The apparatus of claim 1, wherein said apparatus includes a pin movably positioned within said pin guide device, and wherein said apparatus is mounted to a user's finger, and wherein said apparatus is at least partially inserted into an incision in a patient.

8. The apparatus of claim 1, wherein said apparatus defines a single integral plastic member.

9. A surgical guide apparatus comprising a collar expressly adapted to press fittingly mount to a user's finger, a pin guide tube connected to said collar, and a pin movably positioned in said pin guide tube.

10. The apparatus of claim 9, wherein said pin guide device further defines a hollow tubular pin guide device.

11. The apparatus of claim 9, wherein said pin defines at least one of a K-wire, a fixation device, and a marking device.

12. The apparatus of claim 9, wherein said apparatus is press fittingly mounted to a user's finger.

13. The apparatus of claim 9, wherein said apparatus is mounted to a user's finger, and wherein said apparatus is at least partially inserted into an incision in a patient.

14. The apparatus of claim 9, wherein said apparatus defines a single integral plastic member.

15. A method of inserting a pin comprising mounting a surgical guide apparatus to a user's finger, inserting said finger with said surgical guide apparatus mounted thereon into an incision in a patient such that said finger comes substantially into tactile feedback contact with a bone of said patient, guidingly moving said pin via said guide apparatus such that said pin comes into contact with said bone of said patient, and inserting said pin a distance into said bone of said patient.

16. The method of claim 15, wherein said guide apparatus further defines a collar connected to a substantially tubular pin guide device.

17. The method of claim 15, wherein said apparatus defines a single integral plastic apparatus.

18. The method of claim 16, wherein said guidingly moving said pin via said guide apparatus further defines guidingly moving said pin through said tubular pin guide device.

19. The method of claim 15, wherein said finger includes a glove worn thereon.

20. The method of claim 15, wherein said method includes moving said finger along said bone a predetermined offset distance prior to said inserting said pin into said bone.
